(19) United States
(12) Patent Application Publication
(10) Pub. No.: US 2024/0374884 A1
(43) Pub. Date: Nov. 14, 2024

(54) Microneedle Apparatus And System For Perforation Of The Round Window Membrane

(71) Applicant: The Trustees of Columbia University in the City of New York, New York, NY (US)

(72) Inventors: Aykut AKSIT, New York, NY (US); Jeffrey W. KYSAR, New York, NY (US); Anil K. LALWANI, New York, NY (US); Stephen Leong, New York, NY (US)

(21) Appl. No.: 18/782,298

(22) Filed: Jul. 24, 2024

Related U.S. Application Data
(63) Continuation of application No. PCT/US2023/061545, filed on Jan. 30, 2023.
(60) Provisional application No. 63/304,327, filed on Jan. 28, 2022.
(60) Provisional application No. 63/335,989, filed on Apr. 28, 2022.
(60) Provisional application No. 63/405,329, filed on Sep. 9, 2022.

(51) Int. Cl. A61M 37/00; A61B 1/227; A61M 1/00

(52) U.S. Cl. CPC A61M 37/0015; A61B 1/227; A61M 1/84; A61M 2037/0023; A61M 2037/0061; A61M 2210/0668

(57) ABSTRACT

A microneedle system for use with a micro-endoscope having a suction tubing coupled to a camera scope portion, the system including a needle assembly comprising: a microneedle defining a tip and a base; a support member having a distal end and a proximal end, the microneedle base mounted on the distal end of the support member, and flexible tubing, the proximal end of the support member affixed to the flexible tubing; wherein the suction tubing defines an interior lumen, a bend at the distal end portion thereof and a distal edge that is non-orthogonal to the linear axis of the tubing, such that the distal end portion is visible within the camera field of view, and wherein the needle assembly is received within the interior lumen of the suction tubing such that the microneedle is extendable from the distal end of the suction tubing and visible within the camera field of view.

### CROSS-REFERENCE TO RELATED APPLICATION(S)

This application is a continuation of International Application No. PCT/US2023/061545, filed Jan. 30, 2023; which claims priority to U.S. Provisional Application 63/304,327, filed Jan. 28, 2022; U.S. Provisional Application 63/335,989 filed Apr. 28, 2022; and U.S. Provisional Application 63/405,329, filed Sep. 9, 2022. All foregoing applications are incorporated herein by reference in their entireties for any and all purposes.

### GOVERNMENT RIGHTS

This invention was made with government support under grant DC014547 awarded by the National Institutes of Health. The government has certain rights in the invention.

### FIELD

The disclosed subject matter relates to a microneedle apparatus for perforation of anatomic tissues. More particularly, the subject matter relates to a micro-endoscope for microneedle mediated perforation of the round window membrane of a subject.

### BACKGROUND

Minimally invasive intracochlear access, the first step in inner ear drug delivery, has remained an elusive goal. The round window membrane (RWM) serves as an attractive target as it is the only non-osseus barrier through which the inner ear can be accessed. Current methods for drug delivery include application of hydrogels and nanoparticles to the round window niche for diffusion across the membrane, with or without micro perforations to improve diffusion; pump or catheter implantation; ultrasound induced microbubble cavitation; and direct injection through the RWM. Applications requiring transport across intact RWM result in variable delivery of drugs to the perilymph, while injections across the membrane bypass this barrier and result in higher concentrations within the perilymph.

The RWM is selective in its diffusive properties, and its thickness can vary from anatomy to anatomy. Therefore, passive diffusion through it is both limited in rate, and highly variable, making correct dosing difficult. For large molecule delivery applications, such as vector or micro-RNA delivery, delivery becomes near impossible. Moreover, the middle ear space is complicated.

Accordingly, the current methods of access are inherently imprecise and can result in functional damage to the auditory and vestibular systems. Thus, there is a need for a medical apparatus to provide access across anatomic membranes that is safe, reliable and predictable.

### SUMMARY

In one aspect, the disclosed subject matter provides a microneedle system for use with a micro-endoscope having a camera scope portion and an endoscope camera positioned at a distal end of the camera scope portion, the camera defining an axis and a field of view. The microneedle system includes a needle assembly and a suction tubing. The needle assembly includes a microneedle defining a tip and a base, a support member having a distal end and a proximal end, the microneedle base mounted on the distal end of the support member, and flexible tubing, the proximal end of the support member affixed to the flexible tubing. A suction tubing is coupled to the camera scope portion, the suction tubing defines an interior lumen, a bend at the distal end portion thereof and a distal edge that is non-orthogonal to the linear axis of the tubing, such that the distal end portion is visible within the camera field of view, and wherein the needle assembly is received within the interior lumen of the suction tubing such that the microneedle is extendable from the distal end of the suction tubing and visible within the camera field of view.

In some embodiments, the microneedle has a diameter in the range of 10 μm to 1 mm. In some embodiments, the microneedle has a diameter of 100 μm. In some embodiments, the microneedle is fabricated from photoresin. In some embodiments, the microneedle is synthesized using two-photon polymerization (2PP) lithography. In some embodiments, the microneedle is fabricated from biocompatible polymers, stainless steel, or titanium.

In some embodiments, the support member is a metallic tube. In some embodiments, the support member is a 24-gauge stainless steel tube.

In some embodiments, the flexible tubing is fabricated from polyimide. In some embodiments, the suction tubing has a bend of 30-60 degrees.

In some embodiments, the microneedle includes an internal lumen for injection or aspiration of fluid. In some embodiments, the flexible tubing, the support member and the lumen of the microneedle are in fluid communication.

In some embodiments, a spring mechanism is coupled to the needle assembly to allow for actuation and retraction of the needle assembly within the suction tubing.

In another aspect, the disclosed subject matter provides a microneedle system including a needle assembly including a microneedle defining a tip and a base; a support member having a distal end and a proximal end, the microneedle base mounted on the distal end of the support member, and flexible tubing, the proximal end of the support member affixed to the flexible tubing. A micro-endoscope is provided including a camera scope portion having a distal end; an endoscope camera positioned at the distal end of the scope portion, the camera defining an axis and a field of view; a suction tubing defining an interior lumen and coupled to the camera scope portion, the suction tubing defining a bend at the distal end portion thereof and a distal edge that is non-orthogonal to the linear axis of the tubing, such that the distal end portion is visible within the camera field of view, wherein the needle assembly is received within the interior lumen of the suction tubing such that the microneedle is extendable from the distal end of the suction tubing and visible within the camera field of view.

In some embodiments, the microneedle has a diameter in the range of 10 μm to 1 mm. In some embodiments, the microneedle has a diameter of 100 μm. In some embodiments, the microneedle is fabricated from photoresin. In some embodiments, the microneedle is synthesized using two-photon polymerization (2PP) lithography. In some embodiments, the microneedle is fabricated from biocompatible polymers, stainless steel, or titanium.

In some embodiments, the support member is a metallic tube. In some embodiments, the support member is a 24-gauge stainless steel tube.

In some embodiments, the flexible tubing is fabricated from polyimide. In some embodiments, the suction tubing has a bend of 30-60 degrees.

In some embodiments, the microneedle includes an internal lumen for injection or aspiration of fluid. In some embodiments, the flexible tubing, the support member and the lumen of the microneedle are in fluid communication.

In some embodiments, a spring mechanism is coupled to the needle assembly to allow for actuation and retraction of the needle assembly within the suction tubing.

In another aspect, the disclosed subject matter provides a method for perforating RWM of a subject including providing a needle assembly having a microneedle defining a tip and a base; a support member having a distal end and a proximal end, the microneedle base mounted on the distal end of the support member, and flexible tubing, the proximal end of the support member affixed to the flexible tubing; housing the needle assembly in an interior lumen of a suction tubing coupled to a micro-endoscope, the suction tubing defining a bend at the distal end portion thereof such that the distal end portion of the needle assembly is visible by a camera supported by the micro-endoscope. The middle ear is accessed with the microneedle assembly via a tympanomeatal flap. The microneedle assembly is advanced from the suction tubing to perforate the RWM with the tip of the microneedle. Perforation of the RWM is confirmed by the camera supported by the micro-endoscope.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is simplified representation of the anatomy of the human ear.

FIG. 2(a) is a cross-sectional schematic view of microneedle system in a first position in accordance with an exemplary embodiment of the disclosed subject matter.

FIG. 2(b) is a cross-sectional schematic view of microneedle system in another position in accordance with an exemplary embodiment of the disclosed subject matter.

FIG. 2(c) is a cross-sectional schematic view of microneedle system in a further position in accordance with an exemplary embodiment of the disclosed subject matter.

FIG. 3(a) is a side view of the microneedle system of FIGS. 2(a)-(c) with the microneedle apparatus in a first position.

FIG. 3(b) is a side view of the microneedle system of FIGS. 2(a)-(c) with the microneedle apparatus in a second position.

FIG. 4(a) is a view of the microneedle apparatus in the position of FIG. 2(a) when viewed from an endoscope.

FIG. 4(b) is a view of the microneedle apparatus in the position of FIG. 2(c) when viewed from an endoscope.

FIG. 5(a) is a perspective view of an actuator mechanism of the microneedle system in accordance with exemplary embodiments of the disclosed subject matter.

FIG. 5(b) is a side view of the actuator mechanism of the microneedle system of FIG. 5(a).

FIG. 5(c) is a sectional view of the actuator mechanism taken along lines 5-5 of FIG. 5(b)

FIG. 5(d) is a sectional view of an actuator mechanism of the microneedle system in accordance with another exemplary embodiment of the disclosed subject matter.

FIG. 6 is a light microscope image of the microneedle system.

FIGS. 7(a)-(b) are endoscopic images of the microneedle device within the middle ear. FIG. 7(a) is an image of a microneedle extending toward the round window niche. FIG. 7(b) is an image of a microneedle perforating the round window membrane.

FIGS. 8(a)-(b) are endoscopic images of the round window membrane (RWM) prior to perforation by the microneedle apparatus. FIG. 8(a) is a pre-perforation RWM viewed head-on. FIG. 8(B) is a pre-perforation RWM viewed with a posterior-facing angle.

FIGS. 8(c)-(d) are endoscopic images of the RWM following perforation by the microneedle apparatus. FIG. 8(C) is a post-perforation RWM viewed head-on. Arrow indicates the perforation. FIG. 8(D) is a post-perforation RWM viewed with a posterior-facing angle.

### DETAILED DESCRIPTION

It is to be understood that both the foregoing general description and the following detailed description are exemplary and explanatory only, and are not restrictive of the invention, as claimed. In this description, the use of the singular includes the plural, the word “a” or “an” means “at least one,” and the use of “or” means “and/or,” unless specifically stated otherwise. Furthermore, the use of the term “including,” as well as other forms, such as “includes” and “included” is not limiting. Also, terms such as “element” or “component” encompass both elements or components comprising one unit and elements or components that comprise more than one unit unless specifically stated otherwise.

Use of the term “about,” when used with a numerical value, is intended to include +/−10%. For example, if a dimension is identified as about 200, this would include 180 to 220 (plus or minus 10%).

The terms “patient,” “individual,” and “subject” are used interchangeably herein, and refer to a mammalian subject to be treated, with human patients being preferred. In some cases, the methods of the invention find use in experimental animals, in veterinary application, and in the development of animal models for disease.

A microneedle apparatus is disclosed herein that facilitates application of microneedles to anatomic tissues, such as the RWM, using a transcanal approach aided by endoscopy.

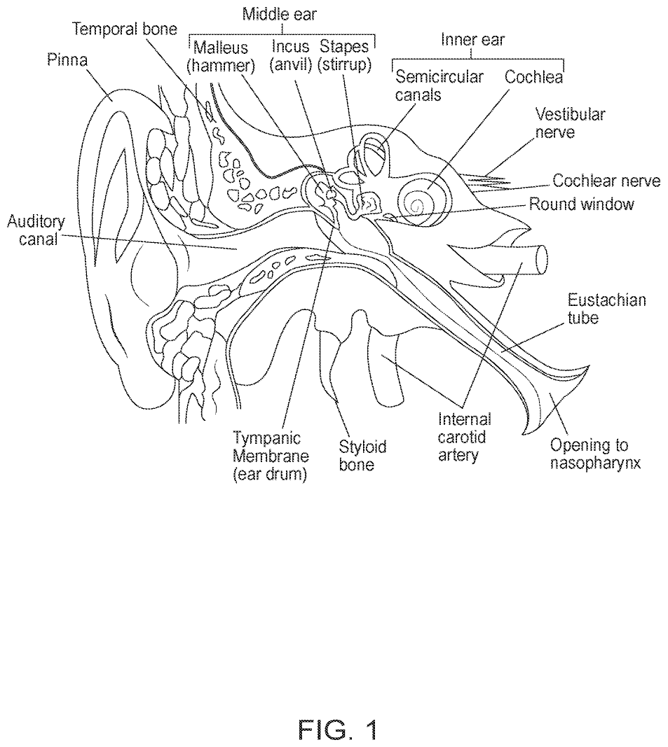

As shown in FIG. 1, the anatomy of the ear includes a middle ear comprising the hammer, anvil, and stirrup bones, and an inner ear comprising the semicircular canals and cochlea. The middle ear and inner ear have barriers to entry and are separated from auditory canal by the tympanic membrane or ear drum. Moreover, the inner ear is further protected from entry by its almost impenetrable structure. The RWM (secondary tympanic membrane) disposed at the inner ear provides an avenue to permit local delivery of therapeutic agents directly to the inner ear.

The RWM is a three-layered structure designed to protect the inner ear from middle ear pathology and facilitate active transport. There is an outer epithelial layer that faces the middle ear, a central connective tissue layer, and an inner epithelial layer interfacing with the scala tympani. The most prominent feature of the outer epithelial layer is the extensive interdigitations and tight junctions of its cells; in addition, there is also a continuous basement membrane layer. This architecture with tight junctions and a continuous basement membrane functions as a defensive shield designed to protect the inner ear from middle ear infections. The connective tissue core contains fibroblasts, collagen, and elastic fibers, and houses blood and lymph vessels. The connective tissue is divided roughly into thirds differing in fiber type and density thus essentially establishing a gradient. This layer is responsible for providing compliance to the RWM. Finally, there is a discontinuous inner epithelial layer that bathes in the perilymph of the scala tympani. As previously noted, conventional transtympanic delivery is limited as it relies on the ability of particles to diffuse or be actively transported across this three layered membrane.

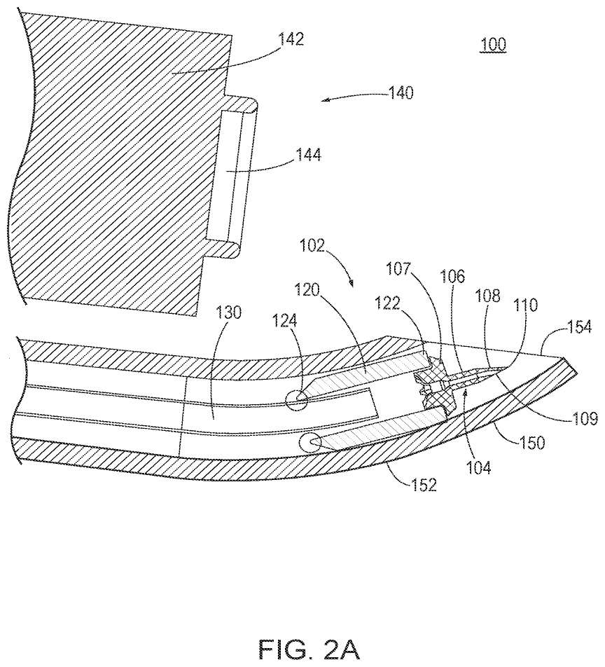

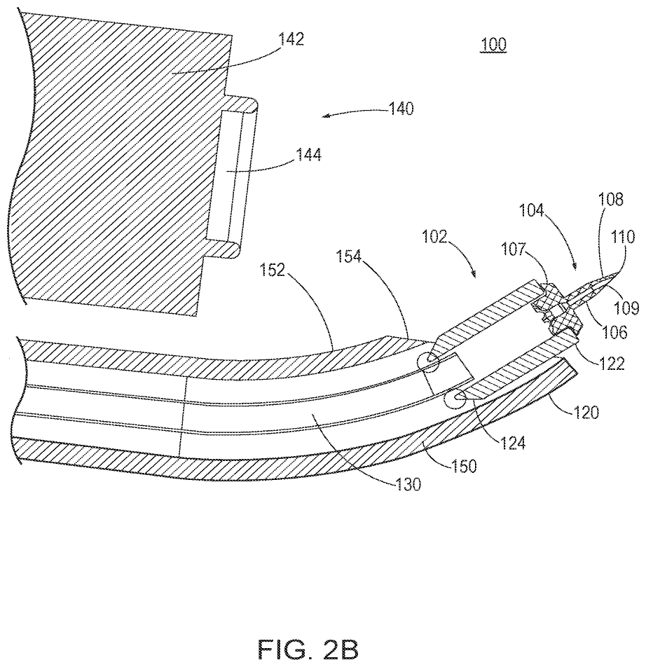

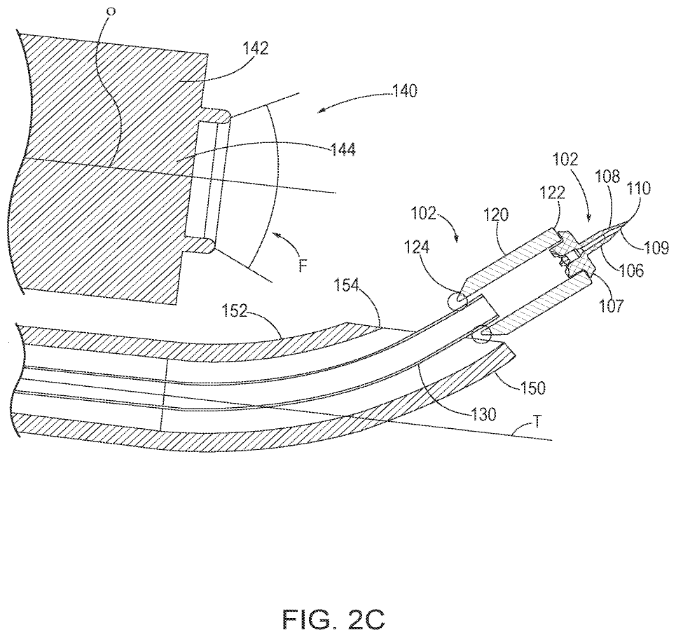

Microneedle System. The microneedle system 100 illustrated in FIGS. 2(a)-(c) and 6, and described herein allows for the deployment of one or more microneedles 104 for perforation of tissue, such as RWM perforation. The microneedle system 100 includes a needle assembly 102 and a micro-endoscope 140. The micro-endoscope 140 includes a camera scope portion 142 and a suction tubing 150. An endoscope camera 144 is positioned at a distal end of the scope portion 142. As shown in FIG. 2(c), the camera optics define an optical axis O and a field of view F (not shown to scale). The suction tubing 150 is coupled to the scope portion 142. In some embodiments, the suction tubing 150 defines a bend 152 at the distal end portion thereof that is non-orthogonal to the linear axis T of the tubing 150. The distal edge 154 is provided at a non-orthogonal angle to the local linear axis of the tubing 150 at the distal edge. Due to the bend 152 in the suction tubing and/or an angle cut in the distal edge 154, the tip 110 of the microneedle 104 is provided with improved visibility within the camera field of view.

The microneedle assembly 102 includes a microneedle 104 mounted on the blunt distal end 122 of a support member 120. In some embodiments, the support member 102 is a metallic tube, such as stainless steel. The proximal end 124 of the support member 120 is mounted to flexible tubing 130. The microneedle assembly 102 is disposed in the interior lumen of the suction tubing 150 such that a portion of the microneedle assembly 102 is extendable from the distal end 154 of the suction tubing 150. The bend portion 152 in the suction tubing 150 improves the visibility of the microneedle 104 within the camera field of view. An actuation system, e.g., spring mechanism 160, allows for the actuation and retraction of the needle assembly within the suction tubing.

Microneedle. As illustrated in FIGS. 2(a)-(c), the microneedle system 100 includes a microneedle assembly 102 including one or more microneedles 104. In an exemplary embodiment, one microneedle 104 is used, and in some embodiments, an array of microneedles may be provided at the distal portion of the assembly 102. The microneedle 104 is fabricated in some embodiments using two-photon polymerization (2PP) lithography with an acrylic-based resin, such as negative-tone resins, IP photoresins, e.g., IP-S Photoresist. In some embodiments, biocompatible polymers, stainless steel, or titanium can be used to manufacture the microneedle. The microneedle 104 can be a hollow microneedle including an enlarged base portion 107, a substantially cylindrical shaft 106, a substantially conical tapered portion 108 and a sharpened tip portion 110. The microneedle 104 can be provided with one or more interior lumens 109 including an opening proximal the tip 110 for injection/introduction or aspiration of fluid. In some embodiments, the lumen 109 is in fluid communication with an interior portion of the support member 120 and the interior lumen of the flexible tubing 130 to allow for infusion of fluid from the needle 104 to the tissue or to allow aspiration of fluid from the tissue.

In some embodiments, the microneedle 104 is manufactured from ultra-high precision 3D molds made via 2PP lithography. Two-photon lithography can be used to manufacture molds for making thermoplastic microneedle arrays for drug delivery and fluid sampling across the anatomic membranes the ear, eye and the CNS such as the RWM. Since the precision of this manufacturing process is very high, very smooth ultra-sharp needles can be made that are specifically engineered to reduce insertion force, minimizing the damage to the tissue in question and any surrounding tissue. For example, hollow microneedles with a diameter of about 100 μm can be used to perforate the RWM without hearing loss; these perforations have been shown to heal completely within 48-72 hours. Perforations made by the microneedles described herein can be lens-shaped or slit-like in nature and in some cases are generated through separation rather than scission of membrane fibers.

In an exemplary embodiment, the shaft 106 has a diameter of 100 μm. In some embodiments, the shaft has a diameter in the range of 10 μm to 1 mm. Exemplary diameters dimensions of the microneedle 104 include 50 μm, 60 μm, 75 μm, 90 μm, 100 μm, 110 μm, 125 μm, 1400 μm, 150 μm and any dimensions inclusive. In an exemplary embodiment, the needle length is 475 μm. In some embodiments, the needle length is 250 μm, 275 μm, 300 μm, 350 μm, 400 μm, 450 μm, 475 μm, 500 μm, 550 μm, 600 μm, 650 μm, 700 μm, 750 μm and any dimensions inclusive. In an exemplary embodiment, the diameter of the interior lumen 109 is 30 μm. In some embodiments, diameter of the interior lumen 109 is 15 μm, 20 μm, 30 μm, 40 μm, 50 μm, 60 μm, 70 μm, 80 μm and any dimensions inclusive. In an exemplary embodiment, the sharpness of the needle is defined by a tip radius of 500 nm to 3 μm. In some embodiments, the tip radius is 500 nm, 600 nm, 700 nm, 800 nm, 900 nm, 1 μm, 2 μm, 3 μm and any dimensions inclusive. Further details regarding the microneedle are disclosed in applications WO/2014/093875; U.S. Pat. No. 10,821,276; WO/2015/20092; U.S. Pat. No. 11,413,191; U.S. application Ser. No. 17/887,966; WO/2017/160948; US 2019/0200927; WO/2019/136133; US 2020/0345994; WO/2019/204760; US 2021/0045925; US 2022/0175413; WO/2021/050404; US 2022/0176096; WO/2020/214802; and US 2022/0032023, all of which are incorporated by reference in their entirety herein.

Support member. The microneedle 104 is mounted on the blunt distal end 122 of support member 120. In an exemplary embodiment, the support member 120 is a 2.5 mm long 24-gauge stainless-steel cylindrical tube. In some embodiments, the support member is a 27-gauge tube. The microneedle 104 is fixed to the support member 120, e.g., using resin epoxy (Gorilla Glue, Inc., OH) to provide a leakproof seal between the lumen 109 and the interior of the support member 120.

Flexible tubing. The proximal end 124 of the support member 120 is affixed to flexible tubing 130. An O-ring or other securement can be provided to provide a leakproof seal between the interior of the flexible tubing 130 and the interior of the support member 120. In an exemplary embodiment, the flexible tubing 130 is a polyimide tubing (Avantor, Radnor, PA) compatible with injection and aspiration. The flexible tubing 130 provides actuation to the microneedle assembly 102, also enabling aspiration or injection through the tubing 130 while inserted into the inner ear space. In an exemplary embodiment, the polyimide tube (200 μm inside diameter, 240 μm outside diameter) was attached to the end of the support member 120, e.g., a 24-gauge stainless steel tube, and fed through the suction tubing 150. Polyimide tubing material provides high strength, good biocompatibility, and chemical inertness, which make it an ideal conduit for direct drug delivery and sampling. Polyimide tubing further provides high flexibility. The low bending modulus is not only useful for turning the bend inside the suction tube 150, but also provided great practical convenience for the stability of the device during injection or aspiration. In some embodiments, the bend is 45 degrees. In some embodiments, the bend is about 30 degrees to about 60 degrees.

Micro-endoscope. The microneedle assembly 102, including the microneedle 104, the support member 120 and the flexible tubing 130, is threaded into the interior lumen of the suction tubing 150 used with micro-endoscope 140. In an exemplary embodiment, the micro-endoscope 140 is a Colibri ENT micro-endoscope, specifically altered to provide safety to the microneedle and the patient, as well as adequate visualization of the needle tip 110 (3NT Medical, Short Hills, NJ). The disclosed subject matter ensures placement of the microneedle 104 and micro-endoscope 130 in front of the RWM with great precision and to ensure controlled perforation.

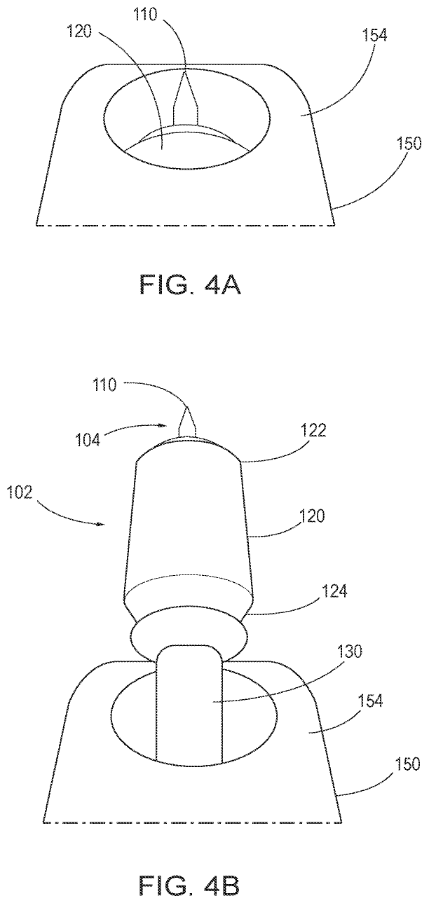

Suction tubing. The suction tubing 150, which houses the microneedle assembly 102 is provided with a curved portion 152, and deployed under full visual guidance by the endoscope camera 144. The curve 152 of the suction tubing 150 enabled going around the bony overhang of the round window and the correct extension of the tube 150 was seen to ensure placement. As shown in FIGS. 2(a)-(c) and 3(a)-(b), the suction tubing 150 includes a bend 152 in order to facilitate such visualization of the microneedle assembly 102. In some embodiments, the bend is about 45 degrees from the linear-axis T of the tubing 150. In some embodiments, the bend is about 30 degrees to about 60 degrees and all positions inclusive. In some embodiments, the bend is about 30 degrees to about 90 degrees and all positions inclusive. For appropriate visualization of the microneedle from the endoscope camera, the Colibri suction tube tip had to be modified. In its default form, it was not possible to visualize the microneedle. Suction tubing was cut at a 40-degree angle, yielding a view simulated in FIGS. 4(a)-(b). FIG. 4(a) is a view of the microneedle apparatus 102 in the position of FIG. 2(a), e.g., the microneedle apparatus 102 viewed from the endoscope 144, when the microneedle apparatus 102 is withdrawn/retracted in the suction tubing 150. FIG. 4(b) is a view of the microneedle apparatus 102 in the position of FIG. 2(c), e.g., the microneedle apparatus 102 viewed from the endoscope 144, when the microneedle apparatus 102 is extended from the suction tubing 150.

The configuration of the microneedle system 100 addresses the delicate nature of the ultra-sharp tip 110 of the microneedle 104 that provides the controlled perforation of the RWM. However, the tip 110 can blunt easily by brittle failure when pressed against a hard surface, such as bone. Accordingly, the tip 110 of the microneedle 104 is protected from damage by retracting the microneedle assembly 102 deep into the suction tubing 150 until properly located at the desired tissue for perforation. In some embodiments, the diameter of the support member 120 is designed specifically to fit into the interior of the suction tubing 150 of the micro-endoscope 140. FIG. 2(a) illustrates the microneedle assembly 102 retracted into the tubing 150 in a “‘protected” state. FIG. 2(b) illustrates that the microneedle assembly 102 is extended from the tubing 150. FIG. 2(c) illustrates that the microneedle assembly 102 is further extended from the tubing 150 for perforation of the RWM.

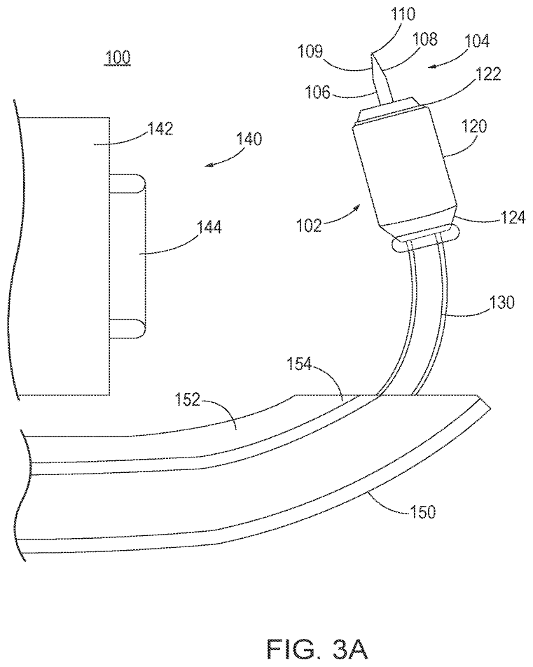

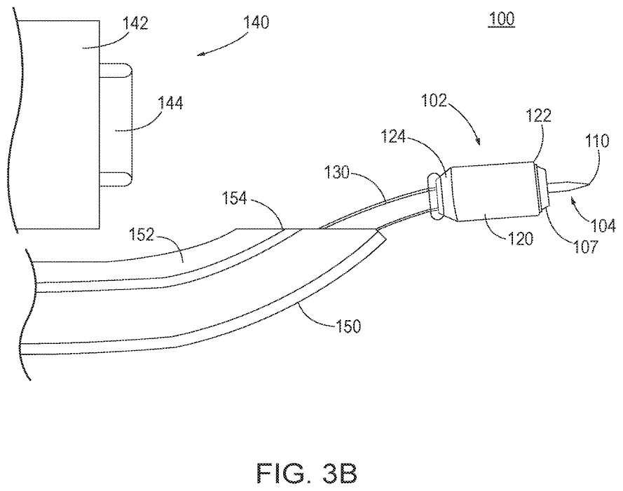

The RWM is a delicate tissue prone to ripping and tearing if manipulated. While the microneedles described herein are safe in perforating the RWM, perforations heretofore have been made with the help of microscale manipulators along with a custom-engineered head holder. A hand-held device, such as microneedle system 100 described herein presents a significant challenge, since even small tremors of the hand can translate to large strains on the membrane while a needle is inserted through. Considering injection and aspiration rates of 1 μL/min or less, the lower bound of what is expected from the device is that it should be stable for more than two minutes. Therefore, an ‘extended’ state of the microneedle actuation system is provided, which allows for large movements of the endoscope that causes minimal strain to the RWM. A schematic demonstration of this phenomenon can be seen in FIGS. 3(a)-(b) . . . . Movement of the microneedle apparatus 102 when microneedle 104 is embedded in the RWM is permitted due to the flexibility of the polyimide tubing 130. The tubing can be seen to be bent in one way in FIG. 3(a) and the other in FIG. 3(b).

Actuation system. To allow for actuation of the microneedle from the distal end of the endoscope device, a spring-loaded actuator 160 is provided that allows for consistent advancement and retraction of the microneedle. In an exemplary embodiment, the actuator 160 is custom 3D-printed (FormLabs, Somerville, MA) a spring-loaded piece that allows for consistent advancement and retraction of the microneedle. This piece was designed to fit snugly underneath the clinician's thumb, such that the mechanism could be ergonomic and straightforward to use. The piece was specifically designed to generate adequate force to perforate the RWM and retract the microneedle immediately after perforation. Custom 3D-printed piece was press-fit into a cavity in the Colibri endoscope. The stainless-steel tube 120, polyimide tubing 130, and 3D-printed thumbpiece 160 were affixed using resin epoxy (Gorilla Glue, Inc., OH).

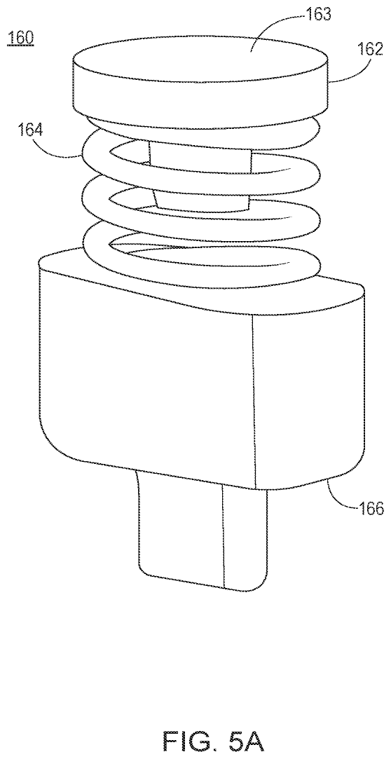

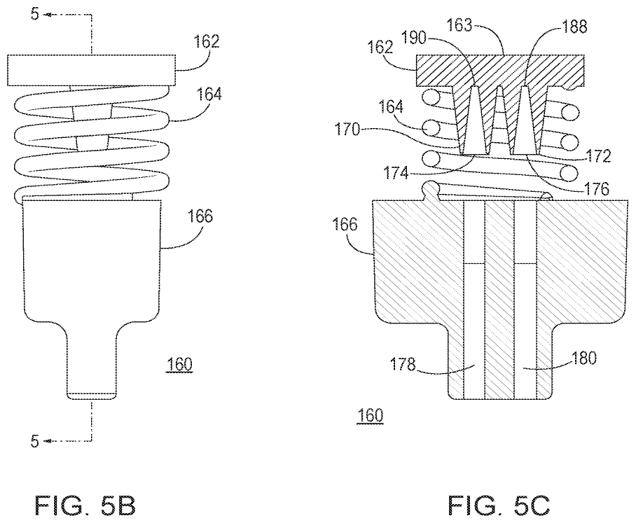

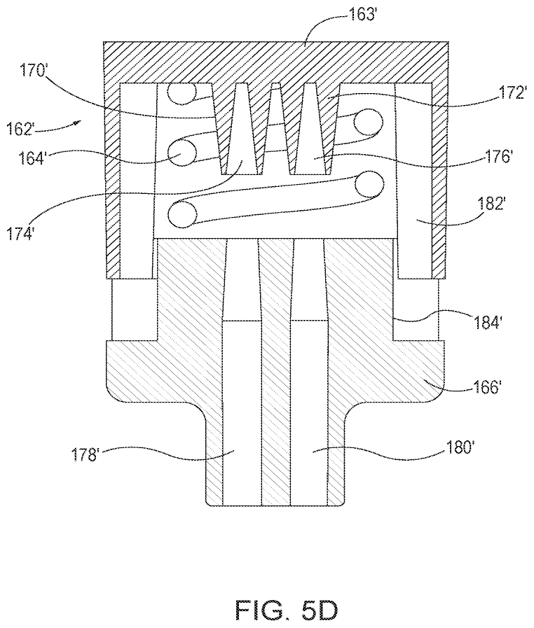

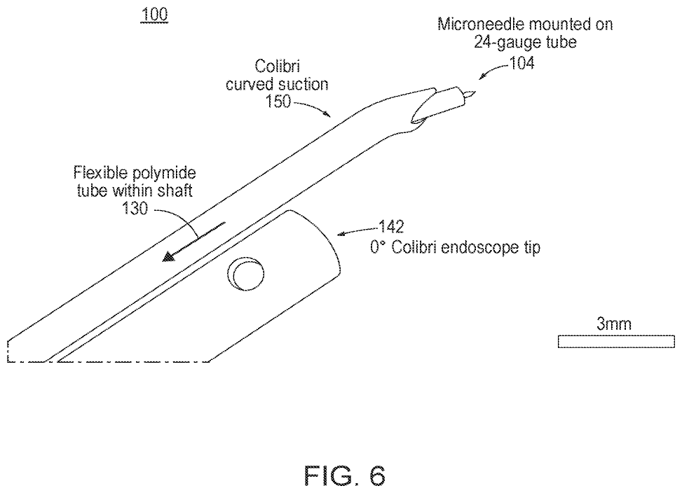

An exemplary embodiments of the actuator 160 is illustrated in FIG. 5(a)-(c). The Actuator 160 includes a button 162 for actuation by a surgeon, an insert 166 for coupling with the flexible tubing 130 and a biasing member 164, such as a coil spring. In some embodiments, the one end of the biasing member is coupled to the button and one end of the biasing member is coupled to the insert 166. The surface 163 of the button 162 is actuated by the surgeon against the bias of the biasing member 164. The button 162 can be advanced distally against the insert 166 coupled to the flexible tubing 130, thereby driving the flexible tubing distally within the suction tubing 150 and advancing the microneedle assembly 102 out of the suction tubing 150, as shown in FIGS. 2(c) and 3(a)-(b). When the button 162 is released, the bias of the biasing member 164 drives the insert 166 and the flexible tubing 130 coupled thereto proximally, and thereby pulls the microneedle assembly 102 proximally towards the suction tubing 150. In some embodiments, the actuator 160 facilitates the introduction of therapeutic fluid from the perforation site via the needle lumen(s) 109 and the aspiration of fluid from the perforation site via lumen(s) 109. As shown in FIG. 5(c), the button 162 includes a pair of projections 170/172, each having a lumens 174/176 respectively extending therein. The lumens 174/176 mate with the lumens 178/180 in the insert when the button 162 is depressed. The lumens 178/180 connect with lumens in the flexible tubing 130 (not shown). In some embodiments, the lumens 174/176 in button 162 are connected to sources of fluid and/or pumps for the introduction and/or withdrawal of fluids. In such embodiments, the end portions 188/190 of lumens 174/176 are connected to such sources of fluid and/or pumps via extensions of lumens 174/176 (not shown).

FIG. 5(d) illustrates a further embodiment of an actuator, designated actuator 160′. Actuator 106′ is substantially identical to actuator 160 with the significant differences noted herein. For example, button 162′ includes a longitudinal extension 182′ slidable with a cooperating longitudinal recess 184′ in the insert 166′ to provide additional stability between the button 162′ and the insert 166′.

The tubing 150 is fastened to the endoscope portion 142 and connected to an actuator 160 provided at the proximal end of the tubing 150. A fixed human cadaveric temporal bone sample was used to evaluate device design for efficacy in visualizing and perforating the RWM.

In some embodiments, the endoscope portion 142 and the suction tubing 150 (exclusive of the distal end portion 152) is substantially straight. In some embodiments, the endoscope portion 142 is continuously curved. In some embodiments, the endoscope portion 142 has one or more bends separated by either straight or curved segments. The endoscope portion 142 can be fabricated from metal, polymer or ceramic. In some embodiments, the shape of the endoscope is modified during the procedure, for example, by use of internal cables or wires to change the configuration. For example, the shape of the endoscope can be modified manually. In some embodiments, the endoscope includes an automatic actuation system, wherein the shape of the endoscope is modified with the automatic actuation system. In some embodiments, the endoscope is configured to bend independently in two orthogonal directions about the longitudinal axis. In some embodiments, one part of the endoscope can be rotated about the longitudinal axis relative to an adjoining segment. In some embodiments, the endoscope is telescoping.

Method of Use. The microneedle assembly 102 works in conjunction with micro-endoscope 130 to facilitate perforation of the RWM with hollow microneedles 104 under full visual guidance, thus supporting the use of microneedles for inner ear diagnosis and intervention in a minimally invasive way. The microneedle system 100 was introduced into the middle ear space of human cadaveric samples after raising a tympanomeatal flap. The tip 154 of the curved tubing 150 was placed in front of the round window niche and the microneedle assembly 102 was deployed from the tip 154 of the tubing 150, thereby advancing the tip 110 of the microneedle 104 to perforate the RWM. After retraction of the microneedle assembly 102 into the tubing 130, RWM perforations were identified via endoscope camera 144. Perforations were inferiorly located and slit-like in shape.

A transcanal approach to access the round window niche was used by Peters et al. via a sialendoscope to visualize the round window niche through a myringotomy. A guide wire was fed through the working channel to prove feasibility of instrumentation of the RWM. Peters, G., et al., Middle-ear endoscopy and trans-tympanic drug delivery using an interventional sialendoscope: feasibility study in human cadaveric temporal bones. J Laryngol Otol, 2010. 124(12): p. 1263-7. Early et al. used a 250 μm metallic microneedle to sample perilymph from a human fresh frozen temporal bone through a transcanal tympanostomy approach which required drilling of the round window overhang. Early, S., et al., A novel microneedle device for controlled and reliable liquid biopsy of the human inner ear. Hear Res, 2019. 381: p. 107761.

By using the novel apparatus and methods described herein, the RWM was accessed via a minimally invasive, transcanal approach which requires no drilling. In order to do so, the microneedle assembly 102 was used in conjunction with the micro-endoscope 150, e.g., the Colibri Micro-ENT Scope developed by 3NT Medical (Rosh HaAyin, Israel). It is understood that any micro-endoscope can be used herein. This single-use endoscope provides an advantage that it is smaller in size relative to other endoscopes and has a 45° curved suction that can be rotated and extended with a thumbpiece. The standard micro-endoscope has been modified in several ways to perforate the RWM, as demonstrated with human cadaveric RWM. Specifically, the Colibri curved suction tubing is modified to house and protect our 100 μm diameter microneedle, actuated by a custom 3D printed spring-loaded button system 160, and direct the microneedle 104 towards the RWM for perforation.

Temporal Bone Preparation Human temporal bones were obtained from the Columbia University Vagelos College of Physicians & Surgeons Anatomy Lab. All cadaveric samples in the anatomy lab were treated with formalin for tissue fixation. Temporal bones were harvested with the modified block method to preserve middle and inner ear structures (Walvekar et al., 2010, Laryngoscope) and stored in a 4° C. refrigerator. One temporal bone with a relatively straight external ear canal was selected to test the utility of the endoscopic microneedle device. A posterior tympanomeatal flap was raised for middle ear access. 0°, 30°, and 45° Hopkins rod telescopes (Karl Storz S E, Tuttlingen, Germany) were used for cleaning and visualization prior to introducing the endoscopic microneedle device. Pre- and post-perforation images of the RWM were taken with the 45° Hopkins rod telescope.

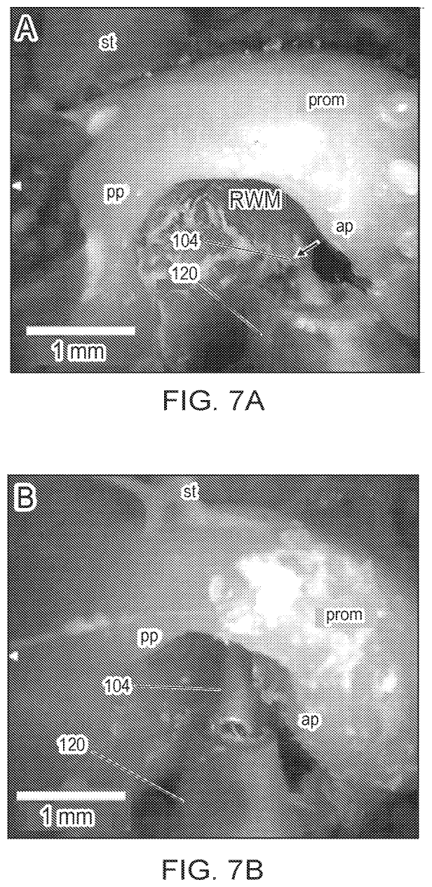

The experiments described herein demonstrate the feasibility of gaining direct inner ear access through the ear canal using microneedles assisted by endoscopy. A tympanomeatal flap was used to reach the middle ear space. Within the middle ear, the micro-endoscope was manipulated such that the distal end 154 (bevel) of the modified curved suction tubing 150 can be angled superiorly at the base of the round window niche (FIG. 7(a)). In FIG. 7(a), the arrow indicates the needle 104. This approach allowed for simultaneous visualization of the inferior edge of the RWM and the tip of the microneedle 104 as it was placed into the round window niche. The microneedle assembly 102 was then advanced by using the actuator 106, e.g., a 3D printed, spring loaded button 106, to perforate the RWM under direct visualization (FIG. 7(b)). With the microneedle safely lodged within the RWM, the endoscope was moved around the round window niche to simulate movements that could take place during injection or aspiration timeframe. The microneedle 104 remained safely lodged within the RWM, thanks to the flexible polyimide tubing 130. (See FIGS. 3(a)-(b).) Anatomical features shown in FIGS. 7(a)-(b) include the cochlear promontory (“prom”); stapes (“st”); round window membrane (“RWM”); posterior pillar (“pp”); and anterior pillar (“ap”).

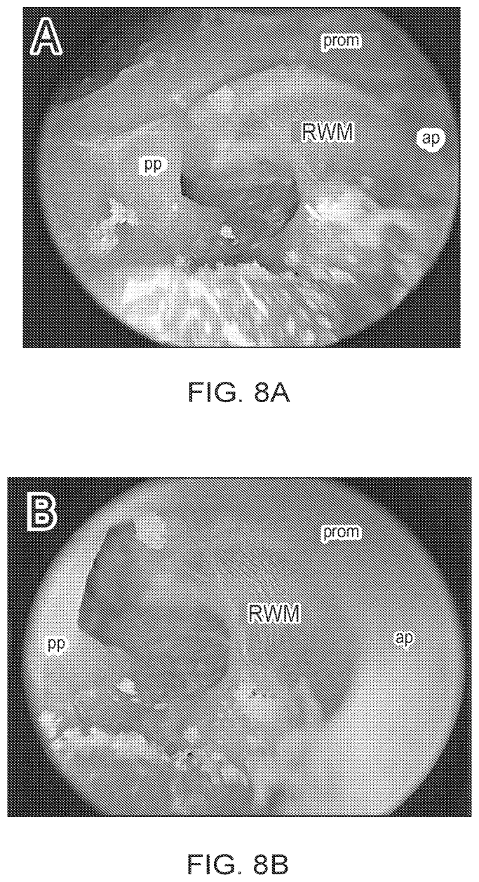

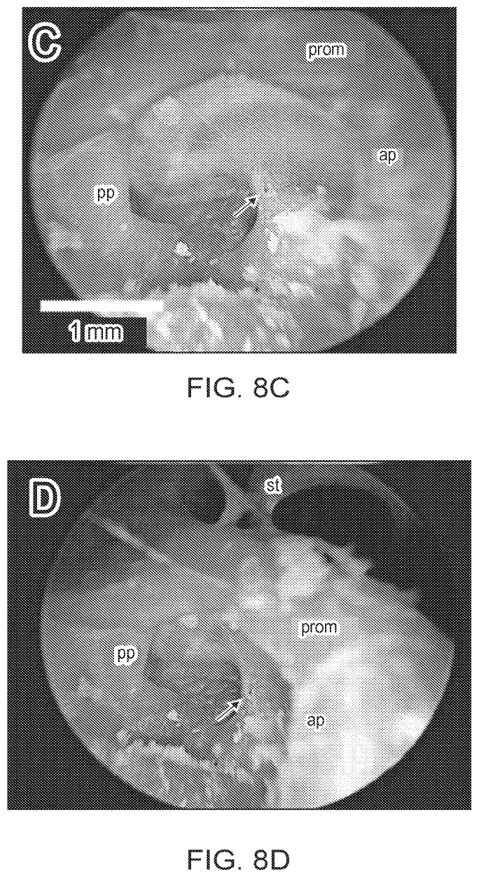

The microneedle assembly 102 was then retracted into the curved suction tubing 150 for protection, and the endoscope 140 was withdrawn from the middle ear. The resulting perforation was located on the inferior aspect of the RWM, was elliptical in nature, and spanned approximately 100 μm (FIGS. 8(a)-(d)), consistent with microneedle-mediated perforations. FIG. 8(a) illustrates pre-perforation RWM viewed head-on. FIG. 8(b) illustrates pre-perforation RWM viewed with a posterior-facing angle. FIG. 8(c) illustrates post-perforation RWM viewed head-on. The arrow indicates the perforation. FIG. 8(d) illustrates post-perforation RWM viewed with a posterior-facing angle. Arrow indicates the perforation. A scale bar in FIG. 8(c) is displayed for reference and refers to FIGS. 8(a)-(d). Anatomical features shown in FIGS. 8(a)-(d) include the cochlear promontory (“prom”); stapes (“st”); round window membrane (“RWM”); posterior pillar (“pp”); and anterior pillar (“ap”).

In accordance with the microneedle system 100 described herein, the utility of a novel endoscopic microneedle application device for transcanal inner ear access is demonstrated. Using a Colibri micro-endoscope mounted with a 100-μm microneedle of the exemplary embodiment, the RWM was successfully perforated after raising a tympanomeatal flap. In some embodiments, the approach described herein does not require any drilling of bone.

The challenge of microneedle stability during injection and aspiration was addressed by over-extending the polyimide tube 130 and compensating the small movements of the hand-held apparatus by the flexibility of the polyimide tube. The small movements were seen to be well compensated by the tubing and well-tolerated by the RWM in the experiment.

The microneedle 104 generated a slit-like perforation. The perforation appears to follow the direction of zero curvature of the RWM. (FIGS. 8(c), (d)).

In some embodiments, metallic microneedles may be used with the microneedle system 100, which could increase durability and decrease the risk of microneedle damage prior to perforation.

In some embodiments, endoscopic microneedle perforations can be performed through a posterior myringotomy.

A microneedle system as described herein is used with a Colibri middle-ear endoscope to perforate the RWM through a transcanal approach. Specifically, the microneedle 104 is housed within a modified curved suction tube 150, which protects the microneedle 104 when not in use. The middle ear is accessed via a tympanomeatal flap, and the microneedle 104 is advanced using a spring-loaded mechanism 160 to perforate the RWM. The resulting perforation is confirmed with direct endoscopic visualization. Thus, the feasibility of minimally invasive, endoscopic microneedle access into the inner ear is demonstrated.

While the work described herein focuses on accessing the cochlea, the technology can be translated to other anatomic barriers and enclosed spaces in the eye and central nervous system. It is to be understood that the claims are not limited to the particular embodiments illustrated above. Various modifications, changes, and variations may be made in the arrangement, operation, and details of the methods and apparatus described above without departing from the scope of the claims.

### REFERENCES

ADDIN EN.REFLIST 1. Szeto, B., et al., Inner ear delivery: Challenges and opportunities. Laryngoscope Investig Otolaryngol, 2020. 5(1): p. 122-131.
2. Salt, A. N. and K. Hirose, Communication pathways to and from the inner ear and their contributions to drug delivery. Hear Res, 2018. 362: p. 25-37.
3. Kelso, C. M., et al., Microperforations significantly enhance diffusion across round window membrane. Otol Neurotol, 2015. 36 (4): p. 694-700.
4. Liao, A. H., et al., Ultrasound-induced microbubble cavitation via a transcanal or transcranial approach facilitates inner ear drug delivery. JCI Insight, 2020. 5(3).
5. Plontke, S. K., et al., Intracochlear Drug Injections through the Round Window Membrane: Measures to Improve Drug Retention. Audiol Neurootol, 2016. 21(2): p. 72-9.
6. Salt, A. N., et al., Marker retention in the cochlea following injections through the round window membrane. Hear Res, 2007. 232(1-2): p. 78-86.
7. Szeto, B., et al., Novel 3D-printed hollow microneedles facilitate safe, reliable, and informative sampling of perilymph from guinea pigs. Hear Res, 2021. 400: p. 108141.
8. Chiang, H., et al., 3D-Printed Microneedles Create Precise Perforations in Human Round Window Membrane in Situ. Otol Neurotol, 2020. 41 (2): p. 277-284.
9. Yu, M., et al., Anatomical and functional consequences of microneedle perforation of round window membrane. Otology & Neurotology, 2020. 41 (2): p. e280-e287.
10. Aksit, A., et al., In-vitro perforation of the round window membrane via direct 3-D printed microneedles. Biomed Microdevices, 2018. 20 (2): p. 47.
11. Peters, G., et al., Middle-ear endoscopy and trans-tympanic drug delivery using an interventional sialendoscope: feasibility study in human cadaveric temporal bones. J Laryngol Otol, 2010. 124 (12): p. 1263-7.
12. Early, S., et al., A novel microneedle device for controlled and reliable liquid biopsy of the human inner ear. Hear Res, 2019. 381: p. 107761.
13. Brenner, M. J., et al., New Medical Device and Therapeutic Approvals in Otolaryngology: State of the Art Review 2020. OTO Open, 2021. 5(4): p. 2473974X211057035.
14. Yu, M., et al., Anatomical and Functional Consequences of Microneedle Perforation of Round Window Membrane. Otol Neurotol, 2020. 41 (2): p. e280-e287.
15. Valentini, C., et al., A Novel 3D-Printed Head Holder for Guinea Pig Ear Surgery. Otol Neurotol, 2021. 42(9): p. e1197-e1202.
16. Aksit, A., et al., Drug delivery device for the inner ear: ultra-sharp fully metallic microneedles. Drug Deliv Transl Res, 2021. 11 (1): p. 214-226.
17. Arriaga, M., et al., Membrane curvature and connective fiber alignment in guinea pig round window membrane. Acta Biomater, 2021. 136: p. 343-362.

What is claimed:

1. A microneedle system for use with a micro-endoscope having a camera scope portion with a distal end and an endoscope camera positioned at the distal end of the camera scope portion, the endoscope camera defining an axis and a field of view, the system comprising:
a needle assembly comprising:
a microneedle defining a tip and a base;
a support member having a distal end and a proximal end, the microneedle base mounted on the distal end of the support member, and
flexible tubing, the proximal end of the support member affixed to the flexible tubing;
a suction tubing coupled to the camera scope portion, the suction tubing defining an interior lumen, a bend at the distal end portion thereof and a distal edge that is non-orthogonal to the linear axis of the tubing, such that the distal end portion is visible within the camera field of view,
wherein the needle assembly is received within the interior lumen of the suction tubing such that the microneedle is extendable from the distal end of the suction tubing and visible within the camera field of view.

2. The microneedle system of claim 1, wherein the microneedle has a diameter in the range of 10 μm to 1 mm.

3. The microneedle system of claim 1, wherein the microneedle has a diameter of 100 μm.

4. The microneedle system of claim 1, wherein the microneedle is fabricated from photoresin.

5. The microneedle system of claim 4, wherein the microneedle is synthesized using two-photon polymerization (2PP) lithography.

6. The microneedle of claim 1, wherein the microneedle is fabricated from biocompatible polymers, stainless steel, or titanium.

7. The microneedle system of claim 1, wherein the support member is a metallic tube.

8. The microneedle system of claim 7, wherein the support member is a 24-gauge stainless steel tube.

9. The microneedle system of claim 1, wherein the flexible tubing is fabricated from polyimide.

10. The microneedle system of claim 1, wherein the curved suction tubing has a bend of about 30 degrees to about 60 degrees.

11. The microneedle system of claim 1, wherein the microneedle includes an internal lumen for injection or aspiration of fluid.

12. The microneedle system of claim 11, wherein the flexible tubing, the support member and the lumen of the microneedle are in fluid communication.

13. The microneedle system of claim 1, further comprising a spring mechanism coupled to the needle assembly to allow for actuation and retraction of the needle assembly within the suction tubing.

14. A microneedle system comprising:
a needle assembly comprising:
a microneedle defining a tip and a base;
a support member having a distal end and a proximal end, the microneedle base mounted on the distal end of the support member, and
flexible tubing, the proximal end of the support member affixed to the flexible tubing;
a micro-endoscope comprising:
a camera scope portion having a distal end;
an endoscope camera positioned at the distal end of the camera scope portion, the camera defining an axis and a field of view;
a suction tubing defining an interior lumen and coupled to the camera scope portion, the suction tubing defining a bend at the distal end portion thereof and a distal edge that is non-orthogonal to the linear axis of the tubing, such that the distal end portion is visible within the camera field of view,
wherein the needle assembly is received within the interior lumen of the suction tubing such that the microneedle is extendable from the distal end of the suction tubing and visible within the camera field of view.

15. The microneedle system of claim 14, wherein the microneedle has a diameter in the range of 10 μm to 1 mm.

16. The microneedle system of claim 14, wherein the microneedle has a diameter of 100 μm.

17. The microneedle system of claim 14, wherein the microneedle is fabricated from photoresin.

18. The microneedle system of claim 17, wherein the microneedle is synthesized using two-photon polymerization (2PP) lithography.

19. The microneedle of claim 14, wherein the microneedle is fabricated from biocompatible polymers, stainless steel, or titanium.

20. The microneedle system of claim 14, wherein the support member is a metallic tube.

21. The microneedle system of claim 20, wherein the support member is a 24-gauge stainless steel tube.

22. The microneedle system of claim 14, wherein the flexible tubing is fabricated from polyimide.

23. The microneedle system of claim 14, wherein the curved suction tubing has a bend of about 30 degrees to about 60 degrees.

24. The microneedle system of claim 14, wherein the microneedle includes an internal lumen for injection or aspiration of fluid.

25. The microneedle system of claim 24, wherein the flexible tubing, the support member and the lumen of the microneedle are in fluid communication.

26. The microneedle system of claim 14, further comprising a spring mechanism coupled to the needle assembly to allow for actuation and retraction of the needle assembly within the suction tubing.

27. A method for perforating the round wind membrane (RWM) of a subject comprising:
providing a needle assembly comprising a microneedle defining a tip and a base; a support member having a distal end and a proximal end, the microneedle base mounted on the distal end of the support member, and flexible tubing, the proximal end of the support member affixed to the flexible tubing;
housing the needle assembly in an interior lumen of a suction tubing coupled to a micro-endoscope, the suction tubing defining a bend at the distal end portion thereof such that the distal end portion of the needle assembly is visible by a camera supported by the micro-endoscope;
accessing the middle ear with the microneedle assembly via a tympanomeatal flap;
advancing the microneedle assembly from the suction tubing to perforate the RWM with the tip of the microneedle; and
confirming the perforation by the camera supported by the micro-endoscope.
